(19) United States
(12) Patent Application Publication
(10) Pub. No.: US 2022/0187309 A1
(43) Pub. Date: Jun. 16, 2022

(54) COMPOSITIONS AND METHODS FOR DETECTION OF DISEASE-RELATED ANTIBODY

(71) Applicants: Sarah E. WHEELER, Bellevue, PA (US); Michael R. SHURIN, Wexford, PA (US); Li LIU, Pittsburgh, PA (US); UNIVERSITY OF PITTSBURGH-OF THE COMMONWEALTH SYSTEM OF HIGHER EDUCATION, Pittsburgh, PA (US); UPMC, Pittsburgh, PA (US)

(72) Inventors: Sarah E. WHEELER, Bellevue, PA (US); Michael R. SHURIN, Wexford, PA (US); Li LIU, Pittsburgh, PA (US)

(21) Appl. No.: 17/607,149

(22) PCT Filed: Apr. 29, 2020
(86) PCT No.: PCT/US2020/030436

§ 371 Date: Oct. 28, 2021

Related U.S. Application Data
(60) Provisional application No. 62/840,699, filed on Apr. 30, 2019.

(51) Int. Cl. G01N 33/68; G01N 33/561

(52) U.S. Cl. CPC G01N 33/6854; G01N 2333/70596; G01N 33/561

(57) ABSTRACT

Disclosed herein are compositions and uses thereof for detection of disease-related antibodies. The methods include contacting a biological sample with a solid support comprising one or more antigens that bind one or more therapeutic monoclonal antibodies, and detecting the disease-related antibody in the biological sample using an electrophoretic method.

### CROSS-REFERENCE TO RELATED APPLICATIONS

This application claims the priority benefit of U.S. Provisional Application No. 62/840,699, filed Apr. 30, 2019, which is incorporated herein by reference in its entirety.

### STATEMENT REGARDING FEDERALLY SPONSORED RESEARCH OR DEVELOPMENT

This invention was made with government support under TR001857 awarded by the National Institutes of Health. The government has certain rights in the invention.

### FIELD

The present disclosure relates to the field of antibody detection.

### BACKGROUND

Multiple myeloma is an incurable hematologic malignancy. Without treatment, typical life expectancy is several months. With current therapy, including immunomodulatory drugs, proteasome inhibitors, cytotoxic agents and autologous hematopoietic stem cell transplant, the survival is usually 4-5 years [Moreau, 2015; Dimopoulos, 2015] with a five-year survival rate about 49%. However, this may be further improved in the near future by the introduction of therapeutic monoclonal antibodies (tmAb) approved by the Food and Drug Administration (FDA) within the past two years, daratumumab and elotuzumab [N. W. C. J. van de Donk, 2012; Raje, 2015; Elsada, 2019; Nooka, 2019; N. W. C .J. van de Donk, 2018]. Daratumumab is a human IgG1/κ tmAb against plasma cell surface antigen CD38, and elotuzumab is a humanized IgG1/κ tmAb targeting a self-ligand receptor, signaling lymphocytic activation molecule family member 7 (SLAMF7) [Nooka, 2019; Costello, 2017]. Due to its efficacy and response rate, daratumumab was recently approved by the FDA as a frontline therapy for newly diagnosed multiple myeloma [Elsada, 2019; FDA, Info of Daratumumab 2018].

As daratumumab gains popularity in treating multiple myeloma, it also complicates the monitoring of myeloma patients. Multiple myeloma is typically monitored by the detection of the disease-associated mAb as an M-protein by protein electrophoresis (SPE) and immunoprecipitation electrophoresis (IEP), as well as the percentage of bone marrow plasma cells, and free light chain ratios [Nooka, 2019; Kyle, 2009; Willrich, 2016; Kumar, 2016].

However, tmAbs such as daratumumab and elotuzumab are also readily detected by SPE and IEP and have electrophoretic properties similar to M-protein [FDA, Info of Daratumumab 2018; Kyle, 2009; Willrich, 2016; Kumar, 2016; McCudden, 2010]. These similarities can result in misclassification of tmAbs as M-protein, and a resultant mischaracterization of treatment efficacy.

More specifically, daratumumab is recommended to be administered weekly for 8 weeks and then biweekly for 16 weeks; finally, at 25 weeks onward, the tmAb is administered every 4 weeks or until disease regression occurs. Elotuzumab is recommended to be given weekly for 8 weeks and then biweekly until disease regression occurs. The half-life of daratumumab is about 21 days. Therefore, during the course of the treatment or several months after the last infusion, there is a high chance that the tmAb is detectable and will interfere with the detection of M-protein on electrophoretic gels. The traditional International Myeloma Working Group defines a complete response to tmAb as no detectable original M-protein in the patients' serum and/or urine by either IEP or SPE. Therefore, substantial responses in patients who received tmAb treatment may be misclassified due to the failure to differentiate between residual disease-related antibodies and tmAbs.

And in fact, it has been shown that without communication to the laboratory of the patient's history of daratumumab therapy, misinterpretation of the therapeutic mAb as endogenous M-protein can occur during the monitoring of plasma cell neoplasms. This includes reporting the level of therapeutic monoclonal antibody as the M-protein when the patient's endogenous M-proteins are below the detection limit; reporting two clones of monoclonal antibody thus leaving a false impression that the patient developed a new clone; and reporting a new monoclonal antibody in light chain disease. A recent study reported that about 11% of total SPE and IEP cases appeared to have daratumumab interference. That study indicated that only 42.5% of the cases with suspected daratumumab due to the characteristic migration pattern were actually taking daratumumab [Liu, 2019]. Therefore, new assays that are able to remove therapeutic mAb or are not subject to the interference of tmAb are needed.

In an effort to mitigate this problem, and since the first report of tmAb interfering with multiple myeloma diagnosis published in 2010, manufacturers of electrophoresis platforms developed assays to remove daratumumab interference using anti-daratumumab specific antisera to treat patient samples. Currently, Sebia's Hydrashift 2/4 daratumumab is the only reagent approved by the FDA to mitigate daratumumab drug interference on Sebia's semi-automated gel platform HYDRASYS 2 [Tang, 2018; FDA, Substantial Equivalence Determination Decision Memorandum: Hydrashift, 2018]. This product is a gel shift assay that uses an anti-daratumumab antibody to form a complex with daratumumab and thereby shift the daratumumab migration pattern during electrophoresis. If a band in question migrates to a different position in the presence of anti-dara antibody, the band is most likely caused by daratumumab [FDA, Substantial Equivalence Determination Decision Memorandum: Hydrashift, 2018]. This assay is specific to daratumumab, but not useful for any other therapeutic mAb. To remove the interference of another tmAb, it will require the development of new antisera and a new FDA approval.

In addition to the Hydrashift assay, mass spectrometry based assays have been used to distinguish tmAbs and M-protein based on their highly accurate molecular mass calculations [Thoren, 2018; Mills, 2015; Moore, 2019]. However, these mass spectrometry based assays are still under development and require expensive equipment and extensive expertise to implement. These assays are not available to most hospital laboratories due to the requirement of expensive equipment and extensive expertise.

What is needed is an assay that is not platform-specific, does not require expensive equipment, and can detect or remove different therapeutic mAbs from various samples or the same sample.

### SUMMARY

The compositions and methods disclosed herein address certain unmet needs in the cancer field. The methods disclosed herein result in surprisingly effective removal of one or more therapeutic monoclonal antibodies from patient sera (e.g., from multiple myeloma patients), regardless of platform. In some embodiments, the method can effectively remove the therapeutic monoclonal antibodies that interfere with the assessment of disease-related antibodies, enabling accurate diagnosis, disease monitoring and determination of remission status in patients being treated with therapeutic monoclonal antibodies.

Disclosed herein are methods of detecting disease-relating antibodies in a biological sample containing or suspected of containing one or more therapeutic monoclonal antibodies comprising: contacting the biological sample with a solid support having one or more antigens bound thereto, wherein the one or more antigens are specific for the one or more therapeutic antibodies, and detecting the disease-related antibody in the biological sample using an electrophoretic method.

In some embodiments, the disease-related antibody comprises an M protein. In some embodiments, the disease-related antibody has a similar electrophoretic mobility to one or more therapeutic monoclonal antibodies, where those therapeutic monoclonal antibodies can bedaratumumab, elotuzumab, isatuximab, tabalumab, indatuximab ravtansin (BT062), denosumab, GSK2857916, or BHQ880. The antigens, which in some embodiments are CD38 or SLAMF7, can be bound to a solid support such as a bead or particle. The biological sample can also be derived from a subject having a plasma cell disorder, such as monoclonal gammopathy of uncertain significance (MGUS), smoldering multiple myeloma (SMM), solitary plasmacytoma, multiple myeloma, waldenstrom's macroglobulinemia (WM), or light chain amyloidosis.

In some aspects, disclosed herein is a kit for removing one or more therapeutic monoclonal antibodies from a biological sample, said kit comprising a solid support and one or more antigens, wherein the one or more antigens are specific for the one or more therapeutic antibodies. The solid support can be a bead or particle. In some embodiments, the one or more antigens are selected from the group consisting of CD38 and SLAMF7.

### DESCRIPTION OF DRAWINGS

FIG. 1 shows an antigen specific antibody depletion assay (ASADA) for daratumumab in saline. Immunofixation results of Daratumumab spiked in saline (0.40 g/L) treated with naïve beads (left panel), and CD38-coated beads with different molar ratios of CD38: daratumumab (right panel).

FIG. 2 (A-D) shows ASADA for daratumumab in serum with different levels of gammaglobulin. Serum protein electrophoresis (FIG. 2A) and immunofixation (FIG. 2B) results of daratumumab (0.40 g/L) spiked in hypo-, normo- and hyper-gammaglobulin serum in the presence of naïve beads (upper panel) and CD38-coated beads (lower panel). Analytical sensitivity of serum protein electrophoresis (FIG. 2C) and immunofixation (FIG. 2D) of daratumumab spiked at 0.20 g/L and 0.80 g/L in hypo-, normo- and hyper-gammaglobulin serum in the presence of naïve beads (upper panel) and CD38-coated beads (lower panel). Arrow: cathodal migration pattern of daratumumab band.

FIG. 3 shows ASADA for elotuzumab in serum with different levels of gammaglobulin. Immunofixation results of elotuzumab (0.40 g/L) spiked in hypo-, normo- and hypergammaglobulin serum in the presence of naïve beads (upper panel) and SLAMF7-coated beads (lower panel). Arrow: elotuzumab migrates in the middle of γ zone.

FIG. 4 shows serum protein electrophoresis of daratumumab depletion in native patient samples. Native patient samples with cathodal IgG/κ bands were used in daratumumab ASADA assay. Ctr, pulldown with antigen naïve control beads. CD38, depletion with CD38-coated beads. 1-21, number of samples. Arrow, the cathodal bands that were tested in the depletion assay. Sample 1 and 16, 2 and 17, 4 and 19, 5 and 20 are four pairs of the sample but tested at two separate runs. Samples from patients under known daratumumab therapy: 1, 2, 3, 4, 6, 8, 10, 16, 17, 18, 19.

FIG. 5 (A & B) shows immunofixation electrophoresis of native patient samples before (FIG. 5A) and after (FIG. 5B) ASADA assay. Arrow, the cathodal IgG/κ bands present in all samples being tested in the ASADA assay (A). 1-21, number of samples. Sample 1 and 16, 2 and 17, 4 and 19, 5 and 20 are four pairs of the sample but tested at two separate runs. Samples from patients under known daratumumab therapy: 1, 2, 3, 4, 6, 8, 10, 16, 17, 18, 19.

FIG. 6 (A & B) shows ASADA for both daratumumab and elotuzumab. Serum protein electrophoresis (FIG. 6A) and immunofixation electrophoresis (FIG. 6B) of CD38 and SLAMF7 double-coated beads deplete daratumumab-(Dara) or elotuzumab-(Elo) spiked serum (bland serum spiked with 0.40 g/L of daratumumab or elotuzumab). Control (Ctr), depletion with naïve beads; Depletion (D), depletion with double-coated beads.

FIG. 7 (A-C) shows IEP of neat and ASADA specimens with co-migrating disease associated monoclonal proteins. Sera from patients not on therapeutic monoclonal antibodies (tmAb) but with disease associated monoclonal proteins that would co-run with tmAb was spiked with 0.40 or 0.80 g/L daratumumab (Dara) or elotuzumab (Elo). Sera without ASADA treatment, with ASADA control treatment, and with tmAb specific ASADA treatment was run on IEP (FIGS. 7A and 7B) and corresponding quantitation of the m-spike in SPE (FIG. 7C).

FIG. 8 shows representative SPE traces of ASADA for daratumumab native patient samples. SPE traces from representative samples of ASADA for daratumumab native patient samples shown in FIG. 4.

### DETAILED DESCRIPTION

Disclosed herein methods compositions, and kits for detecting a disease-related antibody in a biological sample containing one or more therapeutic monoclonal antibodies. The methods include contacting the biological sample with a solid support comprising one or more antigens that bind the one or more therapeutic monoclonal antibodies, and detecting the disease-related antibody in the biological sample using an electrophoretic method. This method has been shown to be surprisingly effective at removing therapeutic monoclonal antibodies from biological samples that interfere with detection of disease and/or treatment efficacy, and more specifically, the detection of disease-related antibodies. As one example, the method is very useful in the detection of M-protein produced during plasma cell disorder, and the reduction of interference between M-protein and therapeutic monoclonal antibodies in electrophoretic detection methods.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

A “control” is an alternative subject or sample used in an experiment for comparison purposes. A control can be “positive” or “negative.”

The terms “antibody” and antibodies” are used herein in a broad sense and include polyclonal antibodies, monoclonal antibodies, and bi-specific antibodies. In addition to intact immunoglobulin molecules, also included in the term “antibodies” are fragments or polymers of those immunoglobulin molecules, and human or humanized versions of immunoglobulin molecules or fragments thereof. Native antibodies are usually heterotetrameric glycoproteins of about 150,000 daltons, composed of two identical light (L) chains and two identical heavy (H) chains. Each heavy chain has at one end a variable domain (V_{H}) followed by a number of constant domains Each light chain has a variable domain at one end (V_{L}) and a constant domain at its other end.

There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired activity. The term “antibody fragment” refers to a portion of a full-length antibody, generally the target binding or variable region. Examples of antibody fragments include Fab, Fab′, F(ab′)₂ and Fv fragments. An “Fv” fragment is the minimum antibody fragment which contains a complete target recognition and binding site. This region consists of a dimer of one heavy and one light chain variable domain in a tight, non-covalent association (V_{H}-V_{L} dimer). It is in this configuration that the three CDRs of each variable domain interact to define an target binding site on the surface of the V_{H}-V_{L} dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “sFv” antibody fragments comprise the V_{H} and V_{L} domains of an antibody, wherein these domains are present in a single polypeptide chain. Generally, the Fv polypeptide further comprises a polypeptide linker between the V_{H} and V_{L} domains which enables the sFv to form the desired structure for target binding.

The antibody fragments, whether attached to other sequences or not, can include insertions, deletions, substitutions, or other selected modifications of particular regions or specific amino acids residues, provided the activity of the fragment is not significantly altered or impaired compared to the nonmodified antibody or antibody fragment. These modifications can provide for some additional property, such as to remove or add amino acids capable of disulfide bonding, to increase its bio-longevity, to alter its secretory characteristics, etc. In any case, the fragment must possess a bioactive property, such as binding activity, regulation of binding at the binding domain, etc. Functional or active regions of the antibody may be identified by mutagenesis of a specific region of the protein, followed by expression and testing of the expressed polypeptide. Such methods are readily apparent to a skilled practitioner in the art and can include site-specific mutagenesis of the nucleic acid encoding the antigen. (Zoller M J et al. Nucl. Acids Res. 10:6487-500 (1982).

Optionally, the antibodies are generated in other species and “humanized” for administration in humans. Humanized forms of non-human (e g , murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

“Disease-related antibody” is used herein to refer to any antibody that has an increased production in a subject due to a disease. One example of a disease-related antibody is an M protein associated with multiple myeloma disease.

“Therapeutic monoclonal antibody” refers to any monoclonal antibody and monoclonal antibody fragment that has been administered to a subject in an effort to treat a disorder or condition.

As used herein, the term “antigen” refers to a molecule that is capable of stimulating an immune response such as by production of antibodies specific for the antigen and fragments thereof comprising an antigenic determinant or epitope. In some embodiments, the antigen is an extracellular region fragment. Antigens of the present invention include CD38 and SLAMF7.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

As used herein, the word “electrophoresis” means the migration of charged molecules in solution in response to an electric field. Their rate of migration depends on the strength of the electric field, the net charge, size and shape of the molecules and the ionic strength, viscosity and temperature of the medium in which the molecules are moving. An “electrophoretic method” is a method that employs electrophoresis. In some embodiments, the electrophoretic method is SDS-PAGE (sodium dodecyl sulphate-polyacrylamide gel electrophoresis). SDS is an anionic detergent that denatures proteins. SDS-PAGE allows for determination of the molecular weight of a polypeptide based on the distance it moves in the electric field under certain conditions. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. One means for determining relative molecular weight of a polypeptide by electrophoresis (Mr) is to plot a standard curve of distance migrated vs. log 10MW for known samples, and determine the logMr of the polypeptide after measuring its distance migrated under the same conditions. “Serum protein electrophoresis” (SPE) refers herein to electrophoresis of polypeptides derived from serum such as antibodies. The term “immunoprecipitation electrophoresis” (IEP) refers to a technique that allows for identification of an electrophoresed polypeptide using an antibody. These are non-limiting examples of electrophoretic methods.

As used herein, “similar electrophoretic mobility” refers to two or more molecules moving a similar distance from one end of an electric field to another end of the electric field under the same conditions (time, temperature, viscosity of the matrix through which the molecules migrate, etc.), wherein “similar” means being indistinguishable to the naked eye. In some embodiments, the two or more molecules have similar electrophoretic mobility wherein one of the molecules is a therapeutic monoclonal antibody and there is no positive control for the therapeutic monoclonal antibody.

As used herein, “M protein” refers to a monoclonal antibody produced by an abnormal plasma cell. An M protein comprises both heavy and light chains, or heavy chains only, or light chains only, and can be of an IgG, IgA, IgM, IgD or IgE subtype with or without corresponding kappa or lambda light chains. In some embodiments, an M protein is an IgG monoclonal antibody. In some embodiments, an M protein is an IgG kappa monoclonal antibody. In some embodiments, an M protein is a kappa monoclonal antibody.

In the present invention, “specific for” and “specificity” mean selective binding. Accordingly, an antibody that is specific for one antigen selectively binds that antigen and not other antigens or not other antigens lacking epitope look-alikes.

The term “subject” includes all mammals. “Mammal” refers to any animal classified as a mammal, including human, domestic and farm animals, nonhuman primates, and zoo, sports, or pet animals, such as dogs, horses, cats, cows, etc.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection.

In some instances, the terms “treat”, “treating”, “treatment” and grammatical variations thereof, include reducing the amount of cancerous cells in a subject, reducing the amount of myeloma cells in a subject, and/or reducing the amount of M-protein in a subject as compared with prior to treatment of the subject or as compared with the incidence of such symptom in a general or study population.

### Methods of Detecting Disease-Related Antibodies

Disclosed herein is a method of detecting a disease-related antibody in a biological sample containing or suspected of containing one or more therapeutic monoclonal antibodies comprising: contacting the biological sample with a solid support having one or more antigens bound thereto, which antigens bind the one or more therapeutic monoclonal antibodies, and detecting the disease-related antibody in the biological sample using an electrophoretic method. As discussed above, this method has been shown to be surprisingly effective at removing therapeutic monoclonal antibodies from biological samples that interfere with detection of disease-related antibodies. Further, coating the magnetic beads with soluble antigens is simple to operate, and the beads (or any solid support) can be used to treat patient serum samples to deplete tmAbs.

As one example, the method is very useful in the detection of an M protein produced during a plasma cell disorder, and in the reduction of interference between M protein and therapeutic monoclonal antibodies in electrophoretic detection methods. Accordingly, the present invention includes embodiments wherein the disease-related antibody is or comprises an M protein associated with multiple myeloma (MM). In that regard, current and future tmAbs have been indicated as promising therapies for MM, however, treatment with tmAbs makes monitoring for MM by SPE and IEP complicated. An accurate estimation of MM therapeutic response by SPE and IEP is a continuous challenge. To mitigate this problem, assays to remove daratumumab interference using anti-daratumumab specific antisera to treat patient samples have been developed. However, to remove the interference of another tmAb, a new antisera will need to be developed and approved by governing agencies. Assays not subject to tmAb interference, such as mass spectrometry assays, are not currently available to most hospital laboratories due to the requirement of expensive equipment and extensive expertise. Therefore, an assay that does not require the development of new antisera, is capable of removing the interference of multiple tmAbs with a single reagent, and/or does not require specialized equipment, is highly desirable. The present invention meets one or more of those needs by providing a solid support such as a bead or particle having bound thereto one or more antigens recognized by tmAbs used in the treatment of multiple myeloma. A biological sample is contacted with the solid support, the solid support and any bound tmABs are removed, and thereafter the constituents remaining in the sample, such as M protein, are analyzed using an electrophoretic method.

However, it should be understood that the present invention encompasses the detection of any disease-related antibody using an electrophoretic method. The disease-related antibody can be of any subtype including an IgG, IgA, IgM, IgD, or IgE subtype. In some embodiments, the disease-related antibody is an IgG antibody. In some embodiments, the disease-related antibody is an IgG kappa antibody. In some embodiments, the disease-related antibody has a far-gamma electrophoretic migration pattern. In other or further embodiments, the disease related antibody has a mid-gamma zone electrophoretic migration pattern. The disease-related antibody can consist of two heavy and two light chains, one or two heavy chains, one light chain, one heavy chain and one light chain, two heavy chains and one light chain, or fragments of any of the aforementioned chain or chains.

In some embodiments, the disorder is a plasma cell disorder. Plasma cell disorders cause increased production of an antibody by a plasma cell. A plasma cell is a differentiated B cell that produces a single type of antibody. Plasma cell disorders include monoclonal gammopathy of uncertain significance (MGUS), smoldering multiple myeloma (SMM), solitary plasmacytoma, multiple myeloma, plasma cell leukemia, waldenstrom's macroglobulinemia (WM), and light chain amyloidosis. Accordingly, in some embodiments, the plasma cell disorder is monoclonal gammopathy of uncertain significance (MGUS). In some embodiments, the plasma cell disorder is smoldering multiple myeloma (SMM). In some embodiments, the plasma cell disorder is solitary plasmacytoma. In some embodiments, the plasma cell disorder is multiple myeloma. In some embodiments, the plasma cell disorder is waldenstrom's macroglobulinemia (WM). In some embodiments, the plasma cell disorder is light chain amyloidosis.

The biological sample containing the disease-related antibody can be of any type. In some embodiments, the sample is a blood sample, a serum sample, a cerebrospinal (CSF) sample or a urine sample. The biological sample is contacted with one or more antigens bound to a solid support, the antigens being specific for one or more therapeutic monoclonal antibodies that are suspected of being in the sample. The biological sample is contacted with the solid support and one or more antigens for a sufficient amount of time to effect binding between any therapeutic monoclonal antibodies in the sample that are specific for the one or more antigens. The solid support is then separated from the sample or a portion of the sample, and the sample or the portion of the sample is analyzed using electrophoretic methods. This method is a surprisingly effective way to remove or reduce therapeutic monoclonal antibodies within the biological sample that interfere with electrophoretic detection of a disease-related antibody. In some embodiments, the therapeutic monoclonal antibodies are reduced to a level in the sample that is undetectable by electrophoreses or an electrophoretic method.

The one or more antigens and the one or more therapeutic monoclonal antibodies can be of any type with the limitation that the one or more antigens are specific for the one or more therapeutic monoclonal antibodies. As described above, “specific for” means that the antigen and therapeutic monoclonal antibody selectively bind one another. This binding can be of a high affinity or a low affinity. In some embodiments, the antigen on the solid support and therapeutic monoclonal antibody in the biological sample bind with high affinity. In some further embodiments, binding between the antigen and the therapeutic monoclonal antibody in vivo is uncommon or of low occurrence due to low affinity, low or reduced antigen expression, low or reduced antigen concentration, microenvironment conditions, steric hindrance, and/or interference.

The present invention also encompasses embodiments wherein different solid supports are bound to different antigens. Accordingly, provided herein is a method of detecting a disease-related antibody in a biological sample containing or suspected of containing one or more therapeutic monoclonal antibodies comprising: contacting the biological sample with two or more solid supports, each having a different antigen bound thereto, which antigens bind the two or more therapeutic monoclonal antibodies, and detecting the disease-related antibody in the biological sample using an electrophoretic method. In some embodiments, one solid support has a CD38 antigen bound thereto and another solid support has a SLAMF7 antigen bound thereto.

In some embodiments, the one or more therapeutic monoclonal antibodies are used for treatment of a plasma cell disorder. The therapeutic monoclonal antibody can be or comprise daratumumab (U.S. Pat. Nos. 9,603,927 and 7,829,673), elotuzumab (U.S. Pat. No. 8,632,772), isatuximab, tabalumab, indatuximab ravtansin (BT062), denosumab, GSK2857916, or BHQ880. In some embodiments, the therapeutic monoclonal antibody is daratumumab. In some embodiments, the therapeutic monoclonal antibody is elotuzumab. In some embodiments, the therapeutic monoclonal antibody is isatuximab. In some embodiments, the therapeutic monoclonal antibody is tabalumab. In some embodiments, the therapeutic monoclonal antibody is indatuximab ravtansin (BT062). In some embodiments, the therapeutic monoclonal antibody is denosumab. In some embodiments, the therapeutic monoclonal antibody is GSK2857916. In some embodiments, the therapeutic monoclonal antibody is BHQ880.

It should be understood that the biological sample can contain or be suspected of containing more than one therapeutic monoclonal antibody. A great advantage of some embodiments of the invention is that multiple therapeutic monoclonal antibodies can be removed or reduced to an undetectable level in the biological sample within the same step by contacting the sample with the solid support having multiple antigens bound thereto that are specific for the multiple therapeutic monoclonal antibodies. Accordingly, in some embodiments, the therapeutic monoclonal antibodies comprise daratumumab and elotuzumab. In some embodiments, the therapeutic monoclonal antibodies are daratumumab and elotuzumab.

Correspondingly, the one or more antigens can be or comprise CD38 and/or SLAMF7—daratumumab is specific for CD38 and elotuzumab is specific for SLAMF7—or a fragment thereof comprising an antigenic determinant or epitope. “CD38” refers herein to a polypeptide that synthesizes and hydrolyzes cyclic adenosine 5′-diphosphate-ribose, and in humans, is encoded by the CD38 gene. In some embodiments, the CD38 polypeptide is that identified in one or more publicly available databases as follows: HGNC: 1667, Entrez Gene: 952, Ensembl: ENSG00000004468, OMIM: 107270, and UniProtKB: P28907. In some embodiments, the CD38 polypeptide comprises the sequence of SEQ ID NO: 1, or a polypeptide sequence having at or greater than about 80%, about 85%, about 90%, about 95%, or about 98% homology with SEQ ID NO: 1, or a polypeptide comprising a portion of SEQ ID NO: 1. The CD38 polypeptide of SEQ ID NO: 1 may represent an immature or pre-processed form of mature CD38, and accordingly, included herein are mature or processed portions of the CD38 polypeptide in SEQ ID NO: 1. In some embodiments, the antigen is a CD38 fragment that is or comprises the sequence of the extracellular domain. In some embodiments, the antigen is a CD38 fragment that is or comprises an antigenic determinant or epitope. In some embodiments, the antigen is a CD38 fragment that is or comprises the sequence of SEQ ID NO: 8, SEQ ID NO: 9, or SEQ ID NO: 10.

“SLAMF7” refers herein to a polypeptide that is a self-ligand receptor of the signaling lymphocytic activation molecule family, and in humans, is encoded by the SLAMF7 gene. In some embodiments, the SLAMF7 polypeptide is that identified in one or more publicly available databases as follows: HGNC: 21394, Entrez Gene: 57823, Ensembl: ENSG00000026751, OMIM: 606625, and UniProtKB: Q9NQ25. In some embodiments, the SLAMF7 polypeptide comprises the sequence of SEQ ID NO: 2, or a polypeptide sequence having at or greater than about 80%, about 85%, about 90%, about 95%, or about 98% homology with SEQ ID NO: 2, or a polypeptide comprising a portion of SEQ ID NO: 2. The SLAMF7 polypeptide of SEQ ID NO: 2 may represent an immature or pre-processed form of mature SLAMF7, and accordingly, included herein are mature or processed portions of the SLAMF7 polypeptide in SEQ ID NO: 2. In some embodiments, the antigen is a SLAMF7 fragment that is or comprises an antigenic determinant or epitope. In some embodiments, the antigen is a SLAMF7 fragment that is or comprises the sequence of the IgC2 domain In some embodiments, the antigen is a SLAMF7 fragment that is or comprises the sequence of the extracellular domain In some embodiments, the antigen is a SLAMF7 fragment that is or comprises the sequence of SEQ ID NO: 11.

Non-limiting examples of other antigens included in the present invention are B-cell activating factor (BAFF), CD138, RANKL, B cell maturation antigen (BCMA), and DKK1. “BAFF” refers herein to a polypeptide that synthesizes and hydrolyzes cyclic adenosine 5′-diphosphate-ribose, and in humans, is encoded by the TNFSF13B gene. In some embodiments, the BAFF polypeptide is that identified in one or more publicly available databases as follows: HGNC: 11929, Entrez Gene: 1067,3 Ensembl: ENSG00000102524, OMIM: 603969, UniProtKB: Q9Y275. In some embodiments, the BAFF polypeptide comprises the sequence of SEQ ID NO: 3, or a polypeptide sequence having at or greater than about 80%, about 85%, about 90%, about 95%, or about 98% homology with SEQ ID NO: 3, or a polypeptide comprising a portion of SEQ ID NO: 3. The BAFF polypeptide of SEQ ID NO: 3 may represent an immature or pre-processed form of mature BAFF, and accordingly, included herein are mature or processed portions of the BAFF polypeptide in SEQ ID NO: 3. In some embodiments, the antigen is a BAFF fragment that is or comprises an antigenic determinant or epitope. “CD138” refers herein to a polypeptide that synthesizes and hydrolyzes cyclic adenosine 5′-diphosphate-ribose, and in humans, is encoded by the SDC1 gene. In some embodiments, the CD138 polypeptide is that identified in one or more publicly available databases as follows: HGNC: 10658, Entrez Gene: 6382, Ensembl: ENSG00000115884, OMIM: 186355, UniProtKB: P18827. In some embodiments, the CD138 polypeptide comprises the sequence of SEQ ID NO:4, or a polypeptide sequence having at or greater than about 80%, about 85%, about 90%, about 95%, or about 98% homology with SEQ ID NO: 4, or a polypeptide comprising a portion of SEQ ID NO: 4. The CD138 polypeptide of SEQ ID NO: 4 may represent an immature or pre-processed form of mature CD138, and accordingly, included herein are mature or processed portions of the CD138 polypeptide in SEQ ID NO: 4. In some embodiments, the antigen is a CD138 fragment that is or comprises an antigenic determinant or epitope.

“RANKL” refers herein to a polypeptide that synthesizes and hydrolyzes cyclic adenosine 5′-diphosphate-ribose, and in humans, is encoded by the TNFSF11 gene. In some embodiments, the RANKL polypeptide is that identified in one or more publicly available databases as follows: HGNC: 11926, Entrez Gene: 8600, Ensembl: ENSG00000120659, OMIM: 602642, UniProtKB: O14788. In some embodiments, the RANKL polypeptide comprises the sequence of SEQ ID NO: 5, or a polypeptide sequence having at or greater than about 80%, about 85%, about 90%, about 95%, or about 98% homology with SEQ ID NO: 5, or a polypeptide comprising a portion of SEQ ID NO: 5. The RANKL polypeptide of SEQ ID NO: 5 may represent an immature or pre-processed form of mature RANKL, and accordingly, included herein are mature or processed portions of the RANKL polypeptide in SEQ ID NO: 5. In some embodiments, the antigen is a RANKL fragment that is or comprises an antigenic determinant or epitope.

“BCMA” refers herein to a polypeptide that synthesizes and hydrolyzes cyclic adenosine 5′-diphosphate-ribose, and in humans, is encoded by the TNFRSF17 gene. In some embodiments, the BCMA polypeptide is that identified in one or more publicly available databases as follows: HGNC: 11913, Entrez Gene: 608, Ensembl: ENSG00000048462, OMIM: 109545, UniProtKB: Q02223. In some embodiments, the BCMA polypeptide comprises the sequence of SEQ ID NO: 6, or a polypeptide sequence having at or greater than about 80%, about 85%, about 90%, about 95%, or about 98% homology with SEQ ID NO: 6, or a polypeptide comprising a portion of SEQ ID NO: 6. The BCMA polypeptide of SEQ ID NO:6 may represent an immature or pre-processed form of mature BCMA, and accordingly, included herein are mature or processed portions of the BCMA polypeptide in SEQ ID NO: 6. In some embodiments, the antigen is a BCMA fragment that is or comprises an antigenic determinant or epitope.

“DKK1” refers herein to a polypeptide that synthesizes and hydrolyzes cyclic adenosine 5′-diphosphate-ribose, and in humans, is encoded by the DKK1 gene. In some embodiments, the DKK1 polypeptide is that identified in one or more publicly available databases as follows: HGNC: 2891, Entrez Gene: 22943, Ensembl: ENSG00000107984, OMIM: 605189, UniProtKB: O94907. In some embodiments, the DKK1 polypeptide comprises the sequence of SEQ ID NO:7, or a polypeptide sequence having at or greater than about 80%, about 85%, about 90%, about 95%, or about 98% homology with SEQ ID NO: 7, or a polypeptide comprising a portion of SEQ ID NO:7. The DKK1 polypeptide of SEQ ID NO: 7 may represent an immature or pre-processed form of mature DKK1, and accordingly, included herein are mature or processed portions of the DKK1 polypeptide in SEQ ID NO: 7. In some embodiments, the antigen is a DKK1 fragment that is or comprises an antigenic determinant or epitope. The one or more antigens described herein are bound to a solid support. As used herein, “solid support” is not limited to a specific type of support. Rather, a large number of supports are available and are known to one of ordinary skill in the art. Solid supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of the desired end use and suitability for various protocols.

In some embodiments, the solid support is or comprises a particle or a bead. In some embodiments, the solid support bead is magnetic. Solid supports comprising particles and beads have been described in the prior art in, for example, U.S. Pat. Nos. 5,084,169, 5,079,155, 473,231, and 8,110,351. The particle or bead size can be optimized for polypeptide separation and can be a size of 4.5 μm, 2.8 μm, 2.7 μm, or 1.0 μm in diameter. In one embodiment the bead is 1.0 μm in diameter.

The one or more antigens can be bound to the solid support using any method. In some embodiments, the one or more antigens are bound to the solid support through a polyhistidine tag (His-tag), such as six histidine residues, added to the antigen at the C- or N-terminus. In these embodiments, the solid support comprises a metal ligand. Accordingly, in some embodiments, the antigen or the fragment thereof is modified. Two non-limiting examples of antigens having a C-terminal polyhistidine tag are His-tag human CD38 protein (Company: Sino Biological, Catalog: 10818-H08H) and His-tag human SLAMF7 protein (Company: Sino Biological, Catalog: 11691-H08H).

In some embodiments, the one or more antigens are at a total approximate concentration on the beads such that the molar ratio of the therapeutic monoclonal antibody to the antigen for which it is specific is approximately 2:1, 1.6:1, or 1:1. It should be understood that “total approximate concentration” does not refer to concentration of antigen per bead, but instead to a concentration of antigen per aggregate of beads used in the methods described herein. Accordingly, in some embodiments, the molar ratio of CD38 and daratumumab is 3:1. In some embodiments, the molar ratio of CD38 and daratumumab is 2.8:1. In some embodiments, the molar ratio of CD38 and daratumumab is 2.6:1. In some embodiments, the molar ratio of CD38 and daratumumab is 2.4:1. In some embodiments, the molar ratio of CD38 and daratumumab is 2.2:1. In some embodiments, the molar ratio of CD38 and daratumumab is 2:1. In some embodiments, the molar ratio of CD38 and daratumumab is 1.8:1. In some embodiments, the molar ratio of CD38 and daratumumab is 1.6:1. In some embodiments, the molar ratio of CD38 and daratumumab is 1.4:1. In some embodiments, the molar ratio of CD38 and daratumumab is 1.2:1. In some embodiments, the molar ratio of CD38 and daratumumab is 1:1. In some embodiments, the molar ratio of SLAMF7 and elotuzumab is 3:1. In some embodiments, the molar ratio of SLAMF7 and elotuzumab is 2.8:1. In some embodiments, the molar ratio of SLAMF7 and elotuzumab is 2.6:1. In some embodiments, the molar ratio of SLAMF7 and elotuzumab is 2.4:1. In some embodiments, the molar ratio of SLAMF7 and elotuzumab is 2.2:1. In some embodiments, the molar ratio of SLAMF7 and elotuzumab is 2:1. In some embodiments, the molar ratio of SLAMF7 and elotuzumab is 1.8:1. In some embodiments, the molar ratio of SLAMF7 and elotuzumab is 1.6:1. In some embodiments, the molar ratio of SLAMF7 and elotuzumab is 1.4:1. In some embodiments, the molar ratio of SLAMF7 and elotuzumab is 1.2:1. In some embodiments, the molar ratio of SLAMF7 and elotuzumab is 1:1.

Accordingly, in some embodiments, the total approximate concentration of CD38 antigen or a fragment thereof in the biological sample is 2.5×10⁻⁶ M, 3×10⁻⁶ M, 3.5×10⁻⁶ M, 4×10⁻⁶M, 4.5×10⁻⁶ M, 5×10⁻⁶ M, 5.5×10⁻⁶ M, 6×10⁻⁶ M, 6.5×10⁻⁶ M, 7×10⁻⁶M, 7.5×10⁻⁶ M, 8×10⁻⁶M, 8.5×10⁻⁶ M, 9×10⁻⁶ M, 9.5×10⁻⁶ M, 1×10⁻⁵ M, 1.5×10⁻⁵ M, 2×10⁻⁵ M, 2.5×10⁻⁵ M, 3×10⁻⁵ M, at 3.5×10⁻⁵ M, 4×10⁻⁵ M, 4.5×10⁻⁵ M, or 5×10⁻⁵ M. In some embodiments, the total approximate concentration of SLAMF7 antigen or a fragment thereof in the biological sample is 1×10⁻⁶ M, 1.5×10⁻⁶ M, 2×10⁻⁶ M, 2.5×10⁻⁶ M, 3×10⁻⁶ M, 3.5×10⁻⁶ M, 4×10⁻⁶M, 4.5×10⁻⁶ M, 5×10⁻⁶M, 5.5×10⁻⁶ M, 6×10⁻⁶ M, 6.5×10⁻⁶ M, 7×10⁻⁶ M, 7.5×10⁻⁶ M, 8×10⁻⁶M, 8.5×10⁻⁶ M, 9×10⁻⁶M, 1×10⁻⁵ M, 1.5×10⁻⁵ M, 2.5×10⁻⁵ M, 3×10⁻⁵ M, 3.5×10⁻⁵ M, 4×10⁻⁵ M, 4.5×10⁻⁵ M, or 5×10⁻⁵ M.

According to the methods described herein, the biological sample is contacted with the one or more antigens bound to the solid support for a sufficient amount of time to allow for binding between any therapeutic monoclonal antibodies in the sample and the one or more antigens. The biological sample can be in contact with the one or more antigens bound to the solid support for approximately 20 minutes, 15 minutes, 10 minutes, or 5 minutes. In some embodiments, the biological sample is in contact with the one or more antigens bound to the solid support for approximately 14 minutes, 13 minutes, 12 minutes, 11 minutes, 10 minutes, 9 minutes, 8 minutes, 7 minutes, or 6 minutes. After this time, the solid support and the biological sample are separated and the biological sample is analyzed using an electrophoretic method. The separation of the solid support and the biological sample reduces the amount of one or more therapeutic monoclonal antibodies originally contained in the biological sample to a level that is undetectable by an electrophoretic method.

As described above, the electrophoretic method can be any that employs electrophoresis. In some embodiments, the electrophoretic method is serum electrophoresis or immunoprecipitation electrophoresis. In some embodiments, the electrophoretic method is serum electrophoresis. In some embodiments, the electrophoretic method is immunoprecipitation electrophoresis. Included herein are methods that reduce the level of a therapeutic monoclonal antibody to a level at or below approximately 25 mg/dL, approximately 20 mg/dL, approximately 15 mg/dL, approximately 10 mg/dL, or approximately 5 mg/dL. In some embodiments, the level of a therapeutic monoclonal antibody is reduced to a level at or below approximately 20 mg/dL, approximately 19 mg/dL, approximately 18 mg/dL, approximately 17 mg/dL, approximately 16mg/dL, approximately 15 mg/dL, approximately 14 mg/dL, approximately 13 mg/dL, approximately 12 mg/dL, approximately 11 mg/dL, approximately 10 mg/dL, approximately 9 mg/dL, approximately 8 mg/dL, approximately 7 mg/dL, approximately 6 mg/dL, or approximately 5 mg/dL.

### Compositions and Kits

Also included herein are compositions and kits for detecting a disease-related antibody in a biological sample containing or suspected of containing one or more therapeutic monoclonal antibodies where the method comprises contacting the biological sample with a solid support having one or more antigens bound thereto, which antigens bind the one or more therapeutic monoclonal antibodies, and detecting the disease-related antibody in the biological sample using an electrophoretic method.

Provided herein are antigen compositions and kits comprising solid support compositions and antigen compositions. In some embodiments, the antigen composition is or comprises CD38. As described above, “CD38” refers herein to a polypeptide that synthesizes and hydrolyzes cyclic adenosine 5′-diphosphate-ribose, and in humans, is encoded by the CD38 gene. In some embodiments, the CD38 polypeptide is that identified in one or more publicly available databases as follows: HGNC: 1667, Entrez Gene: 952, Ensembl: ENSG00000004468, OMIM: 107270, and UniProtKB: P28907. In some embodiments, the CD38 polypeptide comprises the sequence of SEQ ID NO:1, or a polypeptide sequence having at or greater than about 80%, about 85%, about 90%, about 95%, or about 98% homology with SEQ ID NO:1, or a polypeptide comprising a portion of SEQ ID NO:1. The CD38 polypeptide of SEQ ID NO:1 may represent an immature or pre-processed form of mature CD38, and accordingly, included herein are mature or processed portions of the CD38 polypeptide in SEQ ID NO:1. In some embodiments, the antigen is a CD38 fragment that is or comprises the sequence of the extracellular domain. In some embodiments, the antigen is a CD38 fragment that is or comprises an antigenic determinant or epitope. In some embodiments, the antigen is a CD38 fragment that is or comprises the sequence of SEQ ID NO: 8, SEQ ID NO: 9, or SEQ ID NO: 10.

Also provided herein is a composition that is or comprises a SLAMF7 antigen. “SLAMF7” refers herein to a polypeptide that is a self-ligand receptor of the signaling lymphocytic activation molecule family, and in humans, is encoded by the SLAMF7 gene. In some embodiments, the SLAMF7 polypeptide is that identified in one or more publicly available databases as follows: HGNC: 21394, Entrez Gene: 57823, Ensembl: ENSG00000026751, OMIM: 606625, and UniProtKB: Q9NQ25. In some embodiments, the SLAMF7 polypeptide comprises the sequence of SEQ ID NO: 2, or a polypeptide sequence having at or greater than about 80%, about 85%, about 90%, about 95%, or about 98% homology with SEQ ID NO: 2, or a polypeptide comprising a portion of SEQ ID NO: 2. The SLAMF7 polypeptide of SEQ ID NO: 2 may represent an immature or pre-processed form of mature SLAMF7, and accordingly, included herein are mature or processed portions of the SLAMF7 polypeptide in SEQ ID NO: 2. In some embodiments, the antigen is a SLAMF7 fragment that is or comprises an antigenic determinant or epitope. In some embodiments, the antigen is a SLAMF7 fragment that is or comprises the sequence of the extracellular domain In some embodiments, the antigen is a SLAMF7 fragment that is or comprises the sequence of SEQ ID NO: 11.

Also provided herein is a composition that is or comprises a BAFF antigen. “BAFF” refers herein to a polypeptide that synthesizes and hydrolyzes cyclic adenosine 5′-diphosphate-ribose, and in humans, is encoded by the TNFSF13B gene. In some embodiments, the BAFF polypeptide is that identified in one or more publicly available databases as follows: HGNC: 11929, Entrez Gene: 1067,3 Ensembl: ENSG00000102524, OMIM: 603969, UniProtKB: Q9Y275. In some embodiments, the BAFF polypeptide comprises the sequence of SEQ ID NO:3, or a polypeptide sequence having at or greater than about 80%, about 85%, about 90%, about 95%, or about 98% homology with SEQ ID NO: 3, or a polypeptide comprising a portion of SEQ ID NO:3. The BAFF polypeptide of SEQ ID NO: 3 may represent an immature or pre-processed form of mature BAFF, and accordingly, included herein are mature or processed portions of the BAFF polypeptide in SEQ ID NO: 3. In some embodiments, the antigen is a BAFF fragment that is or comprises an antigenic determinant or epitope.

Also provided herein is a composition that is or comprises a CD138 antigen. “CD138” refers herein to a polypeptide that synthesizes and hydrolyzes cyclic adenosine 5′-diphosphate-ribose, and in humans, is encoded by the SDC1 gene. In some embodiments, the CD138 polypeptide is that identified in one or more publicly available databases as follows: HGNC: 10658, Entrez Gene: 6382, Ensembl: ENSG00000115884, OMIM: 186355, UniProtKB: P18827. In some embodiments, the CD138 polypeptide comprises the sequence of SEQ ID NO: 4, or a polypeptide sequence having at or greater than about 80%, about 85%, about 90%, about 95%, or about 98% homology with SEQ ID NO: 4, or a polypeptide comprising a portion of SEQ ID NO: 4. The CD138 polypeptide of SEQ ID NO: 4 may represent an immature or pre-processed form of mature CD138, and accordingly, included herein are mature or processed portions of the CD138 polypeptide in SEQ ID NO: 4. In some embodiments, the antigen is a CD138 fragment that is or comprises an antigenic determinant or epitope.

Also provided herein is a composition that is or comprises a RANKL antigen. “RANKL” refers herein to a polypeptide that synthesizes and hydrolyzes cyclic adenosine 5′-diphosphate-ribose, and in humans, is encoded by the TNFSF11 gene. In some embodiments, the RANKL polypeptide is that identified in one or more publicly available databases as follows: HGNC: 11926, Entrez Gene: 8600, Ensembl: ENSG00000120659, OMIM: 602642, UniProtKB: O14788. In some embodiments, the RANKL polypeptide comprises the sequence of SEQ ID NO:5, or a polypeptide sequence having at or greater than about 80%, about 85%, about 90%, about 95%, or about 98% homology with SEQ ID NO: 5, or a polypeptide comprising a portion of SEQ ID NO: 5. The RANKL polypeptide of SEQ ID NO: 5 may represent an immature or pre-processed form of mature RANKL, and accordingly, included herein are mature or processed portions of the RANKL polypeptide in SEQ ID NO: 5. In some embodiments, the antigen is a RANKL fragment that is or comprises an antigenic determinant or epitope.

Also provided herein is a composition that is or comprises a BCMA antigen. “BCMA” refers herein to a polypeptide that synthesizes and hydrolyzes cyclic adenosine 5′-diphosphate-ribose, and in humans, is encoded by the TNFRSF17 gene. In some embodiments, the BCMA polypeptide is that identified in one or more publicly available databases as follows: HGNC: 11913, Entrez Gene: 608, Ensembl: ENSG00000048462, OMIM: 109545, UniProtKB: Q02223. In some embodiments, the BCMA polypeptide comprises the sequence of SEQ ID NO:6, or a polypeptide sequence having at or greater than about 80%, about 85%, about 90%, about 95%, or about 98% homology with SEQ ID NO: 6, or a polypeptide comprising a portion of SEQ ID NO: 6. The BCMA polypeptide of SEQ ID NO: 6 may represent an immature or pre-processed form of mature BCMA, and accordingly, included herein are mature or processed portions of the BCMA polypeptide in SEQ ID NO: 6. In some embodiments, the antigen is a BCMA fragment that is or comprises an antigenic determinant or epitope.

Also provided herein is a composition that is or comprises a DKK1 antigen. “DKK1” refers herein to a polypeptide that synthesizes and hydrolyzes cyclic adenosine 5′-diphosphate-ribose, and in humans, is encoded by the DKK1 gene. In some embodiments, the DKK1 polypeptide is that identified in one or more publicly available databases as follows: HGNC: 2891, Entrez Gene: 22943, Ensembl: ENSG00000107984, OMIM: 605189, UniProtKB: O94907. In some embodiments, the DKK1 polypeptide comprises the sequence of SEQ ID NO:7, or a polypeptide sequence having at or greater than about 80%, about 85%, about 90%, about 95%, or about 98% homology with SEQ ID NO: 7, or a polypeptide comprising a portion of SEQ ID NO: 7. The DKK1 polypeptide of SEQ ID NO: 7 may represent an immature or pre-processed form of mature DKK1, and accordingly, included herein are mature or processed portions of the DKK1 polypeptide in SEQ ID NO: 7. In some embodiments, the antigen is a DKK1 fragment that is or comprises an antigenic determinant or epitope.

Any of the antigens provided herein can be modified with a label or tag that facilitates the binding of the antigen to a solid support. In some embodiments, the antigen is modified with a polyhistidine tag at the C- or N-terminus. In some embodiments, the antigen comprises six histidines at its C-terminus.

Any of the antigens provided herein can be expressed using expression vectors that comprise nucleic acid sequences which encode the antigens. The nucleic acid sequence can be inserted into the expression vector which contains the necessary elements for the transcription and translation of the inserted coding sequence. Methods which are well known to those skilled in the art can be used to construct expression vectors containing coding sequences and appropriate transcriptional and translational control elements. These methods include in vitro recombinant DNA techniques, synthetic techniques, and in vivo genetic recombination.

Host cells for producing the antigens of any preceding aspects can be prokaryotic or eukaryotic. E. coli is a preferred host cell, but other suitable hosts include Lactococcus lactis, Lactococcus cremoris, Bacillus subtilis, Vibrio cholerae, Salmonella typhi, Salmonella typhimurium, Neisseria lactamica, Neisseria cinerea, Mycobacteria (e.g., M. tuberculosis), yeasts, baculovirus, mammalian cells, etc.

A host cell strain can be chosen for its ability to modulate the expression of the inserted sequences or to process the expressed polypeptide in the desired fashion. Such modifications of the polypeptide include, but are not limited to, acetylation, carboxylation, glycosylation, phosphorylation, lipidation, and acylation. Post translational processing which cleaves a “prepro” form of the polypeptide also can be used to facilitate correct insertion, folding and/or function. Different host cells which have specific cellular machinery and characteristic mechanisms for post translational activities are available from the American Type Culture Collection (ATCC; 10801 University Boulevard, Manassas, Va. 20110-2209) and can be chosen to ensure the correct modification and processing of a foreign protein. See WO 01/98340.

Expression constructs can be introduced into host cells using well-established techniques which include, but are not limited to, transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun” methods, and DEAE- or calcium phosphate-mediated transfection.

Host cells transformed with expression vectors can be cultured under conditions suitable for the expression and recovery of the protein from cell culture. The protein produced by a transformed cell can be secreted or contained intracellularly depending on the nucleotide sequence and/or the expression vector used. Those of skill in the art understand that expression vectors can be designed to contain signal sequences which direct secretion of soluble antigens through a prokaryotic or eukaryotic cell membrane.

Accordingly, in some embodiments, the antigens disclosed herein are made using an expression vector (e.g., E. coli) that comprises a DNA sequence encoding CD38 or a functional fragment thereof with a C-terminal polyhistidine tag. In some embodiments, the CD38 comprises a sequence of SEQ ID NO: 8, SEQ ID NO: 9, or SEQ ID NO: 10.

In some embodiments, the antigen disclosed herein are made using an expression vector (e.g., E. coli) that comprises a DNA sequence encoding SLAMF7 or a functional fragment thereof with a C-terminal polyhistidine tag. In some embodiments, the SLAMF7 comprises a sequence of SEQ ID NO: 11.

Signal export sequences can be included in a recombinantly produced antigen so that the antigen can he purified from cell culture medium using known methods. Alternatively, recombinantly produced antigens can be isolated from engineered host cells and separated from other components in the cell, such as proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified antigens is at least 80% pure; preferably, the preparations are 90%, 95%0, or 99% pure. Purity of the preparations can be assessed by any means known in the art, such as SDS-polyacrylamide gel electrophoresis or RP-HPLC analysis. Where appropriate, mutant Spy0167 proteins can he solubilized, for example, with urea.

Antigens can be synthesized, for example, using solid phase techniques. See, e.g., Merrifield, J. Am, Chem Soc. 85, 2149 54, 1963, Roberge et al., Science 269, 202 04, 1995. Protein synthesis can be performed using manual techniques or by automation. Automated synthesis can be achieved, for example, using Applied Biosystems 431A Peptide Synthesizer (Perkin Elmer). Optionally, fragments of antigens can be separately synthesized and combined using chemical methods to produce a full-length molecule.

Further provided herein are kits comprising one or more antigens and a solid support. Solid supports include silica gels, resins, derivatized plastic films, glass beads, cotton, plastic beads, alumina gels, synthetic antigen-presenting matrices, cells, and liposomes. A suitable solid support may be selected on the basis of the desired end use and suitability for various protocols. In some embodiments, the solid support is or comprises a particle or a bead. In some embodiments, the solid support bead is magnetic. Solid supports comprising particles and beads have been described in the prior art in, for example, U.S. Pat. Nos. 5,084,169, 5,079,155, 473,231, and 8,110,351. The particle or bead size can be optimized for polypeptide separation and can be a size of 4.5 μm, 2.8 μm, 2.7 μm, or 1.0 μm in diameter. In one embodiment the bead is 1.0 μm in diameter.

The kit can comprise one, two, three, four, five or six antigens. In some embodiments the kit comprises two antigens. In some embodiments, the kit comprises a CD38 antigen and a SLAMF7 antigen.

It should be understood that the foregoing relates to preferred embodiments of the present invention and that numerous changes may be made therein without departing from the scope of the invention. The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention and/or the scope of the appended claims All patents, patent applications, and publications referenced herein are incorporated by reference in their entirety for all purposes.

### EXAMPLES

### Example 1: Materials and Methods

Patient samples. Patient samples sent for SPE and IEP to the University of Pittsburgh Medical Center Immunopathology Laboratory were tested in a pulldown assay. Serum samples were collected in serum separator tubes and were processed at the central laboratory with centrifugation at 3000 rpm for 7 minutes at room temperature on the Beckman Coulter automated line. The samples were then delivered to the immunology laboratory for SPE and IEP.

Materials and reagents. Daratumumab (Janssen Pharmaceuticals, Inc. NJ, USA) and elotuzumab (Bristol-Myers Squibb, New York City, N.Y., USA) were purchased from the research pharmacy at UPMC. Daratumumab was in 20 mg/mL solution. Elotuzumab was reconstituted with water to obtain a concentration of 25 mg/mL per manufacturer instructions. Both drugs were stored at 2-8 ° C. Magnetic beads were from Invitrogen (Dynabeads, Catalog No 10104D, Carlsbad, Calif.). His-tag human CD38 protein (Catalog No 10818-H08H) and His-tag human SLAMF7 (11691-H08H) were from Sino Biological Inc. (Wayne, Pa.). To make the His-tag human CD38, a DNA sequence encoding the extracellular domain of human CD38 (NP_001766.2) (Val 43-Ile 300) with a C-terminal polyhistidine tag was expressed. And to make the His-tag human SLAMF7, a DNA sequence encoding the human SLAMF7 (NP_067004.3) extracellular domain (Met 1-Met 226) was expressed, fused with a polyhistidine tag at the C-terminus.

Magnetic bead His-Tag ASADA assay. Magnetic beads were first coated with His-tag CD38 or His-tag SLAMF7, and then the coated beads were utilized to deplete daratumumab and/or elotuzumab in serum or saline samples. The magnetic bead His-Tag depletion assay was performed following the manufacturer's instructions. First, beads were coated with his-tag human CD38 or SLAMF7. During optimization, 100 μl (0.20 g/L) his-tagged human CD38 was used to coat 25 μl, 50 μl, or 100 μl beads to achieve a CD38: daratumumab molar ratio of 1:1, 1.6:1, or 2:1 respectively. Briefly, beads were thoroughly resuspended before transferring to a microcentrifuge tube. The tube was placed on a magnet for 2 minutes then the supernatant was discarded. His-tagged human CD38 or SLAMF7 100 μl (0.20 g/L) in 1× binding buffer were added to the beads and incubated on a roller for 10 minutes at room temperature. The beads were pulled down by placing the tube on a magnet for 2 minutes then the supernatant was discarded. The beads were then washed 4 times with 300 μl wash buffer. After the final wash, 100 μl patient serum, daratumumab or elotuzumab spiked blank serum or saline solution were added to the beads and rotated overnight at room temperature to deplete daratumumab or elotuzumab in the samples. The supernatant serum was then collected for SPE and IEP after placing the tubes on magnetic plate for 2 minutes. Blank beads not coated by CD38 or SLAMF7 were used a control for each sample.

For double coated beads with both CD38 and SLAMF7, 100 μl beads were incubated with his-tagged human CD38 and SLAMF7, 100 μl each (0.20 g/L). The double coated beads were used to deplete either daratumumab or elotuzumab in serum samples.

SPE and IEP. SPE and IEP were performed on the Helena SPIFE 3000 analyzer (Texas, USA) according to manufacturer's protocol with all reagents recommended by the manufacturer (Cat No. 1088, 3460). Helena Electrophoresis Sample Handler was used to automatically dilute and load serum samples (ESH) (Cat No. 1341). Serum total protein was established by using a digital refractometer (Index Instruments U.S., Inc, Model DR-303). Antisera to IgG, IgA, IgM, Kappa and Lambda for IEP are from the SPIFE ImmunoFix Kits.

### Example 2: Optimization of ASADA Assay

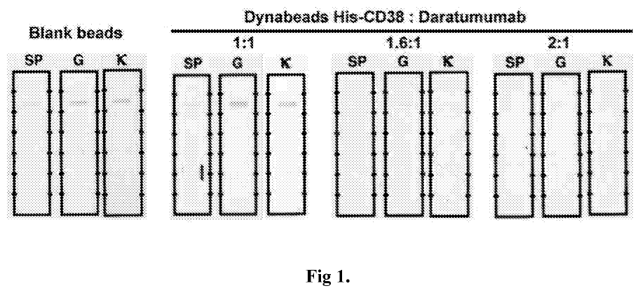

Therapeutic monoclonal antibodies are directed against specific antigens. Leveraging this binding can eliminate monoclonal antibody interference in SPE and IEP. The Antigen Specific therapeutic monoclonal Antibody Depletion Assay (ASADA) was first attempted with daratumumab spiked in saline (0.40 g/L) to clearly visualize the monoclonal bands. Cmax for daratumumab is 0.90 g/L and most patients herein were tested at a minimum after the first half life. Different volumes of beads were used to deplete a fixed amount of daratumumab to find the optimal molar ratio of CD38:daratumumab. As shown in FIG. 1, daratumumab displayed as a clear monoclonal band by SPE and as IgG/κ by IEP when blank beads without coating (or antigen naïve beads) were used in the depletion assay. With CD38-coated beads, the depletion of daratumumab was increasingly complete with the increasing CD38:daratumumab molar ratios. With a 2:1 CD38:daratumumab molar ratio, a complete visible depletion of daratumumab was achieved, while a 1.6:1 molar ratio left a faintly visible band in the IgG lane. Therefore, the 2:1 molar ratio was considered the optimal molar ratio and was used in further experiments.

### Example 3: ASADA Daratumumab or Elotuzumab in Serum

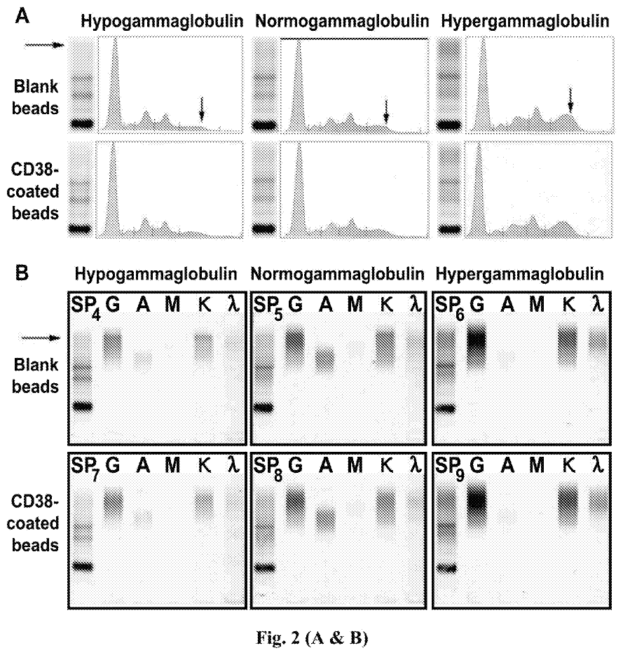

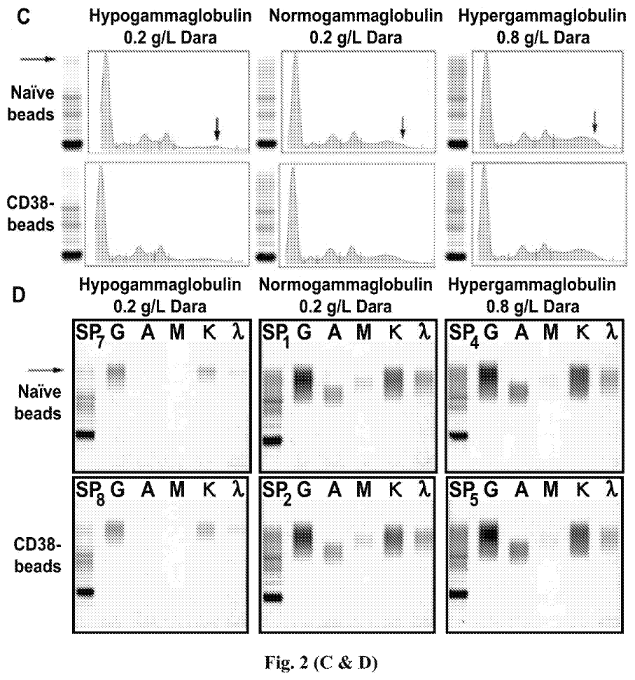

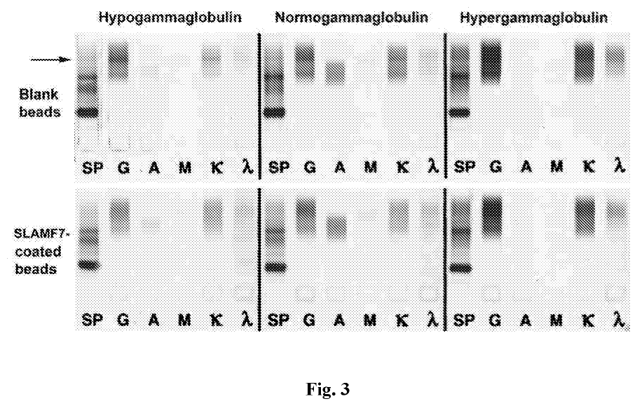

Hypo-, normo- and hyper-gammaglobulin serum spiked with daratumumab or elotuzumab (0.40 g/L) was tested for ASADA assay. FIG. 2 shows that daratumumab at 0.40 g/L in serum exhibited as a light cathodal band by SPE (FIG. 2A) and cathodal IgG/κ by IEP (FIG. 2B) when blank beads (uncoated beads) were used in the pulldown assay as controls. CD38-coated beads with CD38: daratumumab molar ratios at 2:1 completely removed the daratumumab monoclonal band by both SPE and IEP, even in hypogammaglobulin serum where the detection of daratumumab is lower than that of normo- or hyper-gammaglobulin serum. The same results were achieved with elotuzumab spiked serum by both SPE and IEP (FIG. 3).

### Example 4: Sensitivity, Precision, and Specificity of ASADA

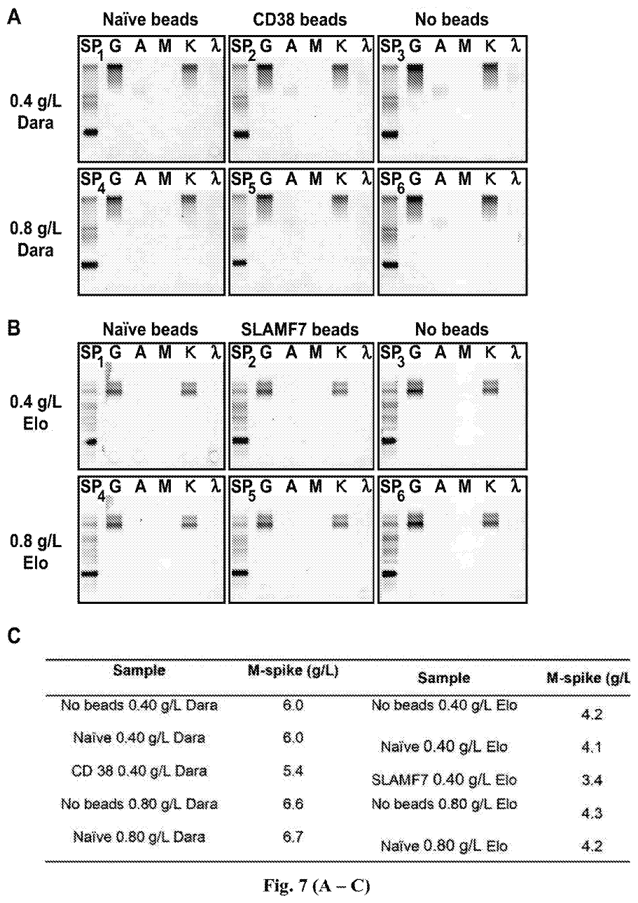

As an assay ASADA introduces a 20% dilution of the sample in its current implementation. Neat samples were further assessed in comparison to naïve and CD38/SLAMF7 ASADA and only minor changes in visual intensity of the SPE and IEP were found (FIGS. 7A-7C). Patients with hyper-, normal, and hypo-gammaglobulin regions were spiked with varying concentrations of daratumumab to assess the sensitivity of ASADA. The data show that daratumumab was visible and specifically removed by ASADA at 0.80 g/L in hypergammaglobulinemia, 0.40 g/L in normal gammaglobulin levels, and 0.20 g/L in hypogammaglobulinemia in SPE (FIG. 2C) and IEP (FIG. 2D).

Precision of ASADA in SPE was assessed by spiking hypergammaglobulinemic and hypogammaglobulinemic patient sera with daratumumab and treating with ASADA for both naïve beads and CD38 beads (Table 1). SPE were quantified using the total protein measurement from the unprocessed sample. Differences between the original sample quantitation and ASADA treated sample quantitation demonstrated clinically acceptable accuracy (Table 1). Analytical specificity was confirmed using sera from patients not on tmAb but with disease associated monoclonal proteins that co-run with tmAb. tmAb was then added to patient sera and samples were treated with ASADA for daratumumab and elotuzumab (FIGS. 7A-7C). Disease associated mAb remained and reductions in M-spike concentration were noted after ASADA

ASADA in the current formulation has a 20% dilution. This has a small effect on the visual intensity of the IEP, allowing for robust visual interpretation. Buffers can be more concentrated allowing for addition of only 1-10% or even 1-5% of the sample volume.

TABLE I

| | | | | | | | M-spike | M-spike |
|---|---|---|---|---|---|---|---|---|
| | | Albumin | Alpha | Alpha | Beta | Gamma | naïve | CD38 |
| | | g/L | 1 g/L | 2 g/L | g/L | g/L | beads | beads |
| Hypergamma | Original sample | 36.4 | 2.3 | 8.9 | 8.8 | 19.7 | | |
| 0.80 g/L Dara | Average (ASADA) | 37.4 | 2.5 | 9.5 | 7.8 | 18.8 | 3.3 | 2.9 |
| | CV | 4% | 6% | 8% | 9% | 5% | 2% | 6% |
| | Original-ASADA | 1.0 | 0.2 | 0.6 | 1.0 | 0.9 | | |
| | | n = 8 | n = 8 | n = 8 | n = 8 | n = 8 | n = 4 | n = 4 |
| Hypogamma | Original sample | 36.0 | 2.4 | 8.8 | 8.2 | 4.6 | | |
| 0.60 g/L Dara | Average (ASADA) | 35.5 | 3.1 | 10.0 | 5.7 | 5.8 | 1.4 [symbol] | 1.1 [symbol] |
| | CV | 3% | 3% | 4% | 8% | 16% | 4% | 4% |
| | Original-ASADA | 0.5 | 0.7 | 1.2 | 2.6 | 1.2 | | |
| | | n = 8 | n = 8 | n = 8 | n = 8 | n = 8 | n = 4 | n = 4 |
| Hypogamma | Original sample | 36.0 | 2.4 | 8.8 | 8.2 | 4.6 | | |
| 0.40 g/L Dara | Average (ASADA) | 35.4 | 3.1 | 10.2 | 5.8 | 5.6 | 1.3 [symbol] | 1.0 [symbol] |
| | CV | 3% | 5% | 5% | 6% | 14% | 6% | 14% |
| | Original-ASADA | 0.6 | 0.7 | 1.4 | 2.4 | 0.9 | | |
| | | n = 8 | n = 8 | n = 8 | n = 8 | n = 8 | n = 4 | n = 4 |

^{a} Measurement range for SPE is not considered quantitatively accurate <2.0 g/L, numeric data is provided here for reference. [symbol]
indicates data missing or illegible when filed

### Example 5: CD38-Coated Beads Pulldown Daratumumab in Native Patient Serum

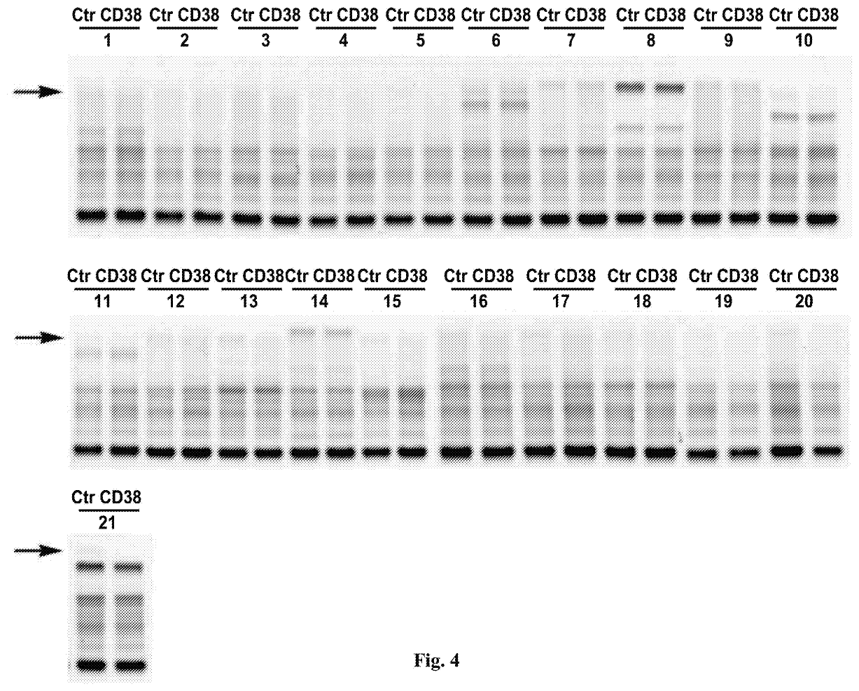

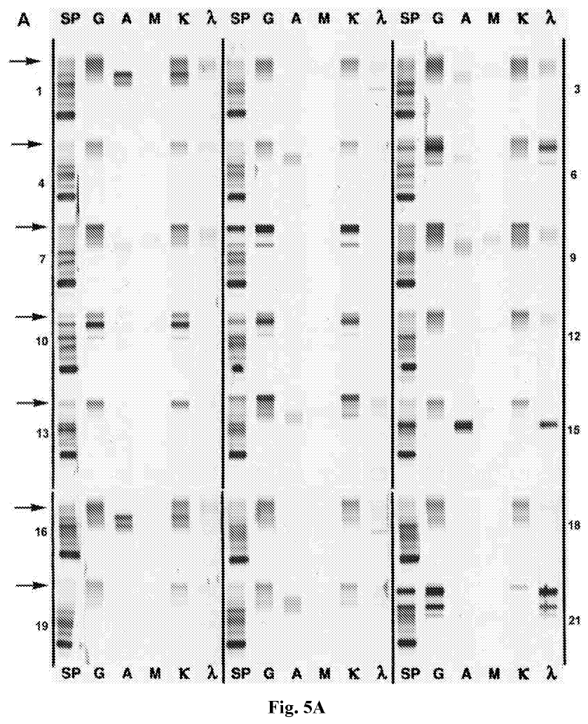

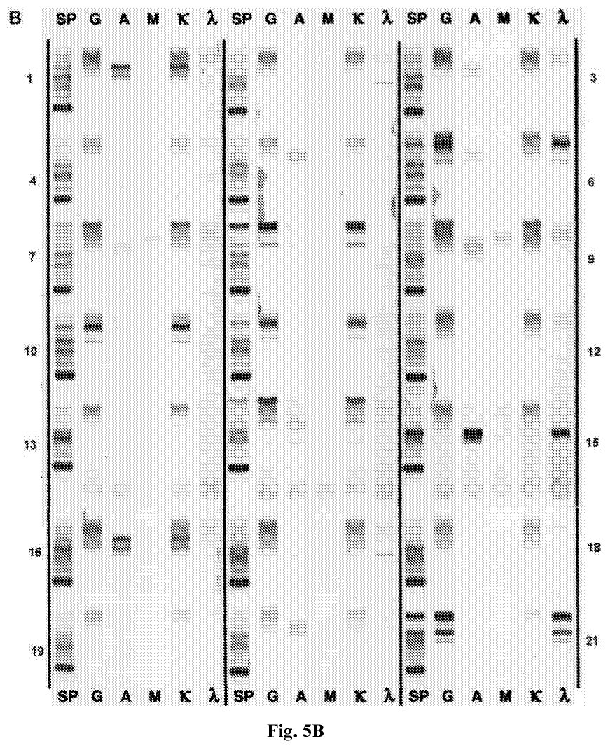

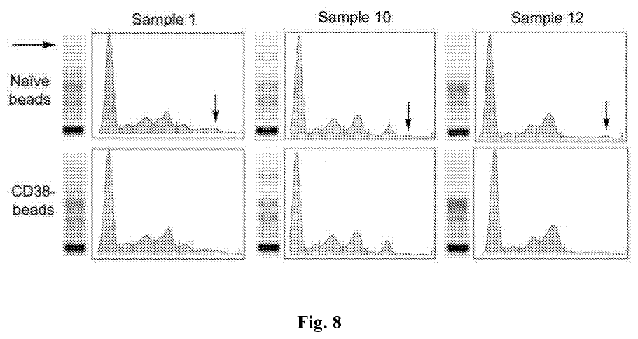

Next, daratumumab ASADA assay was performed in 21 native patient serum samples with known cathodal IgG/κ bands (FIG. 4, and FIGS. 5A and 5B). SPE (FIG. 4 and FIG. 8) and IEP (FIGS. 5A and 5B) demonstrated that the cathodal bands completely disappeared after depletion with CD38-coated beads in samples 1-5, 10-13, 15-21. Review of electronic medical records confirmed daratumumab therapy of these patients. In contrast, daratumumab depletion failed to remove the cathodal bands in samples 6, 7, 9, and 14. Chart reviews revealed that these patients were not on daratumumab therapy, proving that these cathodal bands represented endogenous mAbs and appropriate specificity of the assay. The patient of sample 8 was also on daratumumab therapy. However, the heavy endogenous IgG/κ co-migrated with daratumumab causing the persistence of cathodal IgG/κ band after depletion assay. These results proved that the ASADA assay is specific to daratumumab. Also, four samples were re-tested in a second run (samples 1,2,4,5 correspond to 16,17,19,20 respectively), which achieved the same results, confirming the repeatability of the assay. These results prove the feasibility of ASADA assay to remove daratumumab in patient serum samples.

### Example 6: ASADA with Double-Coated Beads for Depleting Daratumumab and Elotuzumab

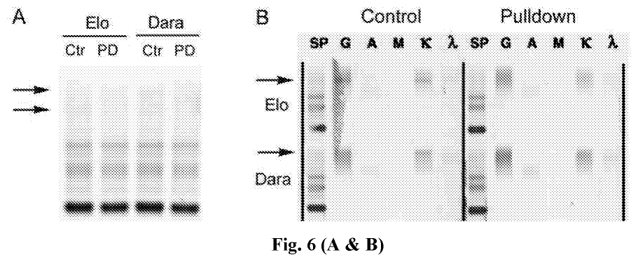

The advantage of ASADA is that it can be multiplexed with multiple antigens used to coat the solid suppport. To test if ASADA can be used as a single assay for depletion of two different mAbs, the beads were coated with CD38 and SLAMF7, to deplete either daratumumab or elotuzumab in serum samples. The double-coated beads depleted either daratumumab or elotuzumab in spiked serum (0.40 g/L) as evidenced by SPE (FIG. 6A) and IEP (FIG. 6B).

### REFERENCES

[1]. P. Moreau, M. Attal, T. Facon, Frontline therapy of multiple myeloma. Blood 125 (2015) 3076-3084.
[2]. M. A. Dimopoulos, P. G. Richardson, P. Moreau, K. C. Anderson, Current treatment landscape for relapsed and/or refractory multiple myeloma. Nat Rev Clin Oncol. 12 (2015) 42-54.

[3]. N. W. van de Donk, S. Kamps, T. Mutis, H. M. Lokhorst, Monoclonal antibody-based therapy as a new treatment strategy in multiple myeloma. Leukemia 26 (2012) 199-213.
[4]. N. Raje, D. L. Longo, Monoclonal antibodies in multiple myeloma come of age. N Engl J Med. 373 (2015) 1264-1266.
[5]. C. Costello, An update on the role of daratumumab in the treatment of multiple myeloma. Ther Adv Hematol. 8 (2017) 28-37.
[6]. Full prescribing information of daratumumab by the FDA. www.accessdata.fda.gov/drugsatfda_docs/label/2018/761036s0141bl.pdf. Revised by FDA in June 2018 (Accessed 11 July 2018).
[7]. R. A. Kyle, S. V. Rajkumar, Criteria for diagnosis, staging, risk stratification and response assessment of multiple myeloma. Leukemia 23 (2009) 3-9.
[8]. M. A. V. Willrich, J. A. Katzmann, Laboratory testing requirements for diagnosis and follow-up of multiple myeloma and related plasma cell dyscrasias. Clin Chem Lab Med. 54 (2016) 907-919.
[9]. C. R. McCudden, P. M. Voorhees, S. A. Hainsworth, H. C. Whinna, J. F. Chapman, et al., Interference of monoclonal antibody therapies with serum protein electrophoresis tests. Clin Chem. 56 (2010) 1897-1899.
[10]. A. E. Axel, C. R. McCudden, H. Xie, B. M. Hall, A. K. Sasser, Development of clinical assay to mitigate daratumumab, an IgGlk monoclonal antibody, interference with serum immunofixation (IFE) and clinical assessment of M-protein response in multiple myeloma. Cancer Res. 74 (2014) Abstract 2563.
[11]. C. McCudden, A. E. Axel, D. Slaets, T. Dejoie, P. L. Clemens, S. Frans, et al., Monitoring multiple myeloma patients treated with daratumumab: teasing out monoclonal antibody interference. Clin Chem Lab Med. 54 (2016) 1095-1104.
[12]. K. Murata, S. I. McCash, B. Carroll, A. M. Lesokhin, H. Hassoun, N. Lendvai, et al., Treatment of multiple myeloma with monoclonal antibodies and the dilemma of false positive M-spikes in peripheral blood. Clin Biochem. 51 (2018) 66-71.
[13]. J. R. Mills, D. L. Murray, Identification of friend or foe: the laboratory challenge of differentiating M-proteins from monoclonal antibody therapies. JALM. An AACC publication 1 (2017) 421-431.
[14]. F. Tang, E. Malek, S. Math, C. L. Schmotzer, R. C. Beck, Interference of Therapeutic Monoclonal Antibodies With Routine Serum Protein Electrophoresis and Immunofixation in Patients With Myeloma: Frequency and Duration of Detection of Daratumumab and Elotuzumab. Am J Clin Pathol. 150 (2018) 121-129.
[15]. FDA, 510(k) Substantial Equivalence Determination Decision Memorandum: Hydrashift, (n.d.). www.accessdata.fda.gov/cdrh_docs/reviews/K172195.pdf (accessed 4 Sep. 2018).
[16]. K. L. Thoren, M. J. Pianko, Y. Maakaroun, C. Ola Landgren, L V Ramanathan, Distinguishing Drug from Disease by Use of the Hydrashift 2/4 Daratumumab Assay. JALM. An AACC publication DOI: 10.1373/jalm.2018.026476
[17]. J. R. Mills, D. R. Barnidge, D. L. Murray, Detecting monoclonal immunoglobulins in human serum using mass spectrometry. Methods 81 (2015) 56-65.
[18]A. Elsada, A. I. Adler, NICE guidance on daratumumab with bortezomib and dexamethasone for previously treated multiple myeloma, Lancet Oncol. 20 (2019) 619-620.
[19]A. K. Nooka, J. L. Kaufman, C. C. Hofmeister, N. S. Joseph, T. L. Heffner, V. A. Gupta, H. C. Sullivan, A. S. Neish, M. V. Dhodapkar, S. Lonial, Daratumumab in multiple myeloma, Cancer 125 (2019) 2364-2382.
[20]N. W. C. J. van de Donk, S. Z. Usmani, CD38 antibodies in multiple myeloma: mechanisms of action and modes of resistance, Front. Immunol. 9 (2018) 2134. doi:10.389/fimmu.2018.02134.
[21]R. A. Kyle, S. V. Rajkumar, Criteria for diagnosis, staging, risk stratification and response assessment of multiple myeloma, Leukemia 23 (2009) 3-9.
[22]S. Kumar, B. Paiva, K. C. Anderson, B. Durie, O. Landgren, P. Moreau, N. Munshi, S. Lonial, J. Bladé, M.-V. Mateos, M. Dimopoulos, E. Kastritis, M. Boccadoro, R. Orlowski, H. Goldschmidt, A. Spencer, J. Hou, W. Joo Chng, S. Z. Usmani, E. Zamagni, K. Shimizu, S. Jagannath, H. E. Johnsen, E. Terpos, A. Reiman, R. A. Kyle, P. Sonneveld, P. G. Richardson, P. McCarthy, H. Ludwig, W. Chen, M. Cavo, J.-L. Harousseau, S. Lentzsch, J. Hillengass, A. Palumbo, A. Orfao, S. Vincent Rajkumar, J. San Miguel, H. Avet-Loiseau, International Myeloma Working Group consensus criteria for response and minimal residual disease assessment in multiple myeloma, 2016, doi:10.1016/51470-2045(16)30206-6.
[23]S. Liu, L., Wertz, W J., Kondisko, A., Shurin, M., Wheeler, Incidence and Management of Therapeutic Monoclonal Antibody Interference in Monoclonal Gammopathy Monitoring, J. Appl. Lab. Med. Accepted (2019).
[24]H. Abramson, Monoclonal antibodies for the treatment of multiple myeloma: an update, Int. J. Mol. Sci. 19 (2018) 3924.
[25]L. M. Moore, S. Cho, K. L. Thoren, MALDI-TOF mass spectrometry distinguishes daratumumab from M-proteins, Clin. Chim. Acta. 492 (2019) 91-94.
[26]P. L. Clemens, X. Yan, H. M. Lokhorst, S. Lonial, N. Losic, I. Khan, R. Jansson, T. Ahmadi, K. Lantz, H. Zhou, T. Puchalski, X. S. Xu, Pharmacokinetics of daratumumab following intravenous infusion in relapsed or refractory multiple myeloma after prior proteasome inhibitor and immunomodulatory drug treatment, Clin. Pharmacokinet. 56 (2017) 915-924.
[27]FDA, PRESCRIBING INFORMATION OF DARATUMUMAB, n.d. https://www.accessdata. fda.gov/drugsatfda_docs/label/2018/761036s0141bl.pdf (accessed Sep. 2, 2019).
[28]L. Rasche, J. Duell, I. C. Castro, V. Dubljevic, M. Chatterjee, S. Knop, F. Hensel, A. Rosenwald, H. Einsele, M. S. Topp, S. Brandlein, GRP78-directed immunotherapy in relapsed or refractory multiple myeloma—results from a phase 1 trial with the monoclonal immunoglobulin M antibody PAT-SM6, Haematologica 100 (2015) 377-384.

1. A method of detecting a disease-related antibody in a biological sample containing one or more therapeutic monoclonal antibodies comprising:
a. contacting the biological sample with a solid support having one or more antigens bound thereto, wherein the one or more antigens are specific for the one or more therapeutic monoclonal antibodies, and
b. detecting the disease-related antibody in the biological sample using an electrophoretic method.

2. The method of claim 1, wherein the disease-related antibody comprises an M protein.

3. The method of claim 1, wherein the one or more therapeutic monoclonal antibodies have a similar electrophoretic mobility to the disease-related antibody.

4. The method of claim 1, wherein the one or more therapeutic monoclonal antibodies comprise an antibody selected from the group consisting of daratumumab, elotuzumab, isatuximab, tabalumab, indatuximab ravtansin (BT062), denosumab, GSK2857916, and BHQ880.

5. The method of claim 1, wherein the one or more therapeutic monoclonal antibodies are selected from the group consisting of daratumumab and elotuzumab.

6. The method of claim 1, wherein the solid support is a bead or particle.

7. The method of claim 1, wherein the one or more antigens are selected from the group consisting of CD38 and SLAMF7.

8. The method of claim 7, wherein the CD38 comprises the amino acid sequence of SEQ ID NO: 8, SEQ ID NO: 9, or SEQ ID NO: 10.

9. The method of claim 7, wherein the SLAMF7 comprises the amino acid sequence of SEQ ID NO: 11.

10. The method of claim 1, wherein the one or more antigens are each at an approximate total concentration of between 1×10⁻⁶ M and 5×10⁻⁵ M on an aggregate of more than one of the solid support.

11. The method of claim 1, wherein the biological sample is a serum sample, a cerebrospinal fluid sample, or a urine sample.

12. The method of claim 1, wherein the biological sample is derived from a subject having a plasma cell disorder.

13. The method of claim 12, wherein the subject is a human.

14. The method of claim 12, wherein the plasma cell disorder is monoclonal gammopathy of uncertain significance (MGUS), smoldering multiple myeloma (SMM), solitary plasmacytoma, multiple myeloma, waldenstrom's macroglobulinemia (WM), or light chain amyloidosis.

15. The method of claim 1, wherein the electrophoretic method is protein electrophoresis or protein immunofixation electrophoresis.

16. A kit for removing one or more therapeutic monoclonal antibodies from a biological sample, said kit comprising a solid support and one or more antigens, wherein the one or more antigens are specific for the one or more therapeutic antibodies.

17. The kit of claim 16, wherein the solid support is a bead or particle.

18. The kit of claim 17, wherein the one or more antigens are selected from the group consisting of CD38 and SLAMF7.

19. The kit of claim 18, wherein CD38 comprises the amino acid sequence selected from SEQ ID NO: 8, SEQ ID NO: 9, or SEQ ID NO: 10.

20. The kit of claim 18, wherein SLAMF7 comprises the amino acid sequence of SEQ ID NO: 2 or SEQ ID NO: 11.

21. The kit of claim 16, wherein the one or more antigens is each at an approximate total concentration of 1×10⁻⁶ M and 5×10⁻⁵ M on an aggregate of more than one of the solid support.

22. The kit of claim 16, wherein the biological sample is a serum sample, a cerebrospinal fluid sample, or a urine sample.

23. The kit of claim 16, wherein the biological sample is derived from a subject having a plasma cell disorder.

24. The kit of claim 23, wherein the subject is a human.

25. The kit of claim 24, wherein the plasma cell disorder is monoclonal gammopathy of uncertain significance (MGUS), smoldering multiple myeloma (SMM), solitary plasmacytoma, multiple myeloma, waldenstrom's macroglobulinemia (WM), or light chain amyloidosis.


SEQUENCE LISTING (11 sequences)

SEQ ID NO: 1 - protein, 300 aa, Homo sapiens
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 3 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 4 - protein, 310 aa, Homo sapiens
MRRAALWLWLCALALSLQPALPQIVATNLPPEDQDGSGDDSDNFSGSGAGALQDITLSQQTPSTWKDTQLLTAIPTSPEPTGLEATAASTSTLPAGEGPKEGEAVVLPEVEPGLTAREQEATPRPRETTQLPTTHLASTTTATTAQEPATSHPHRDMQPGHHETSTPAGPSQADLHTPHTEDGGPSATERAAEDGASSQLPAAEGSGEQDFTFETSGENTAVVAVEPDRRNQSPVDQGATGASQGLLDRKEVLGGVIAGGLVGLIFAVCLVGFMLYRMKKKDEGSYSLEEPKQANGGAYQKPTKQEEFYA

SEQ ID NO: 5 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 6 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 7 - protein, 266 aa, Homo sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 8 - protein, 14 aa, Artificial Sequence
  Synthetic construct
SKRNIQFSCKNIYR

SEQ ID NO: 9 - protein, 14 aa, Artificial Sequence
  Synthetic construct
EKVQTLEAWVIHGG

SEQ ID NO: 10 - protein, 258 aa, Artificial Sequence
  Synthetic construct
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 11 - protein, 226 aa, Artificial Sequence
  Synthetic construct
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM